(19) United States
(12) Patent Application Publication
(10) Pub. No.: US 2012/0129221 A1
(43) Pub. Date: May 24, 2012

(54) BIOCHEMICAL PLATFORM FOR FUELS AND CHEMICALS PRODUCTION FROM CELLULOSIC BIOMASS

(75) Inventors: Zhiliang Fan, Oakland, CA (US); Takao Kasuga, Oakland, CA (US); Weihua Wu, Oakland, CA (US); Xiaochao Xiong, Oakland, CA (US); Ruifu Zhang, Oakland, CA (US)

(73) Assignees: The United States of America, as Represented by the Secretary of Agriculture, Washington, DC (US); The Regents of the University of California, Oakland, CA (US)

(21) Appl. No.: 13/318,340

(22) PCT Filed: Apr. 30, 2010
(86) PCT No.: PCT/US2010/033302

§ 371 Date: Dec. 21, 2011

Related U.S. Application Data
(60) Provisional application No. 61/174,974, filed on May 1, 2009.

(51) Int. Cl. C12P 19/44

(52) U.S. Cl. 435/74

(57) ABSTRACT

An alternative biochemical process for fuels and chemicals production from cellulosic biomass is presented. The method includes an aerobic step where microorganisms convert carbohydrates contained in cellulosic biomass into sugar acids, which can be converted to fuels and chemicals, including ethanol, gluconate, and acetic acid, via fermentation.

This application claims the benefit of U.S. Provisional Application No. 61/174,974, filed May 1, 2009, which is hereby incorporated by reference, in its entirety.

### BACKGROUND

1. Field

The present application relates generally to a method for the conversion of cellulosic biomass into fuels and chemicals; more specifically, it relates to the conversion of cellulosic materials into sugar acids or their salts, which may then be fermented into chemicals including ethanol, gluconate, and acetic acid.

2. Related Art

Cellulosic biomass, which is available at low cost and in large abundance, is one of the only foreseeable sustainable sources for organic fuels, chemicals and materials (1-3). In particular, ethanol production from cellulosic biomass has near-zero greenhouse emissions and offers many other environmental benefits (1, 3, 4). The primary obstacle impeding production of ethanol and other chemicals from cellulosic biomass is the lack of technology for low-cost production (3).

FIG. 1 depicts a traditional biochemical platform or method for fuel- and chemical-production that generates sugars from cellulosic feedstock as reactive intermediates. These sugars can then be fermented to produce fuels and chemicals. There are five key steps involved in the current biochemical platform: (1) pre-treatment, (2) cellulase production, (3) enzymatic hydrolysis, (4) fermentation, and (5) product recovery. The first three steps: pre-treatment, cellulase production, and enzymatic hydrolysis are the three most costly steps in the production process, constituting approximately 65% of the overall processing cost.

The first step, pre-treatment, is a process to remove hemicellulose and lignin to increase the susceptibility of cellulose to subsequent enzymatic hydrolysis, thus allowing the exposed cellulose to be hydrolyzed into sugars fermentable by cellulases. The pre-treatment process tends to be thermo-chemical. Techniques used in the process include treatment with acid or base, or through steam or ammonia explosions. Most of the techniques are energy-intensive, expensive, and often polluting. In addition, capital cost for pre-treatment reactors are extremely high due to specific material requirements for acid or alkali resistance at elevated temperatures.

After the pre-treatment step, cellulases are added in a second step to hydrolyze cellulose, resulting in the production of sugars. While cellulase production costs have dropped significantly due to industrial production of enzymes, costs of this step still remain high. Lowering the processing costs of the two aforementioned steps is crucial for the realization of cost-effective production of ethanol and chemicals from biomass.

### SUMMARY

Herein, an alternative biochemical process for production of fuels and chemicals from cellulosic materials is described. This new approach will utilize natural and recombinant aerobic cellulosic microorganism(s), which secrete enzymes that hydrolyze cellulose and hemicellulose into sugars. Once formed, the majority of the sugars will be oxidized to their corresponding sugar aldonates and will thus be prevented from microbial utilization. Only a small fraction of sugar will be utilized to support cell growth and enzyme production. In a second step, sugar acids (instead of the sugars) will be utilized by natural or recombinant anaerobic microorganism(s) as feedstock to produce ethanol or other chemicals via fermentation.

### DESCRIPTION OF DRAWING FIGURES

FIG. 1 depicts a biochemical platform known in the art for fuel- and chemical-productions (5).

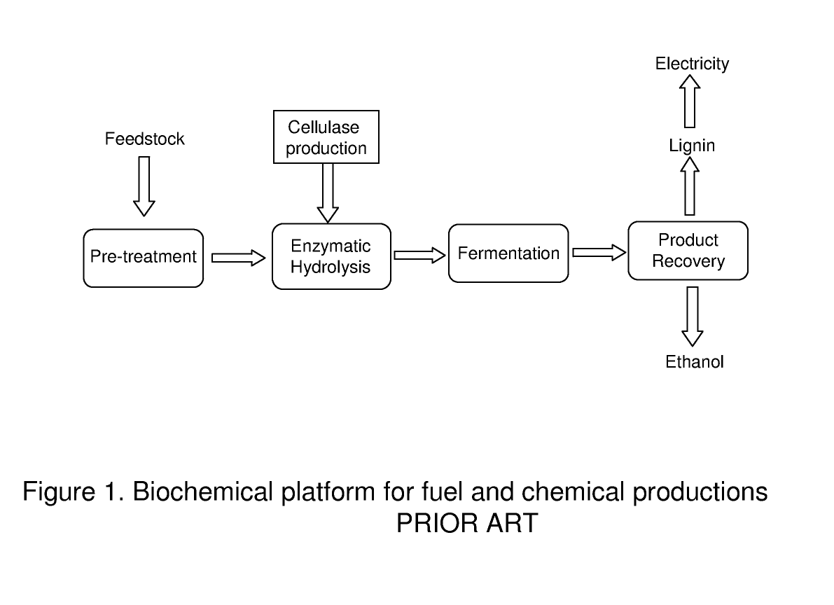

FIG. 2a depicts a biochemical platform disclosed herein for fuel- and chemical-productions by conversion of cellulosic biomass into sugar aldonic acids, which are then fermented to produce energy, biofuels, and chemicals.

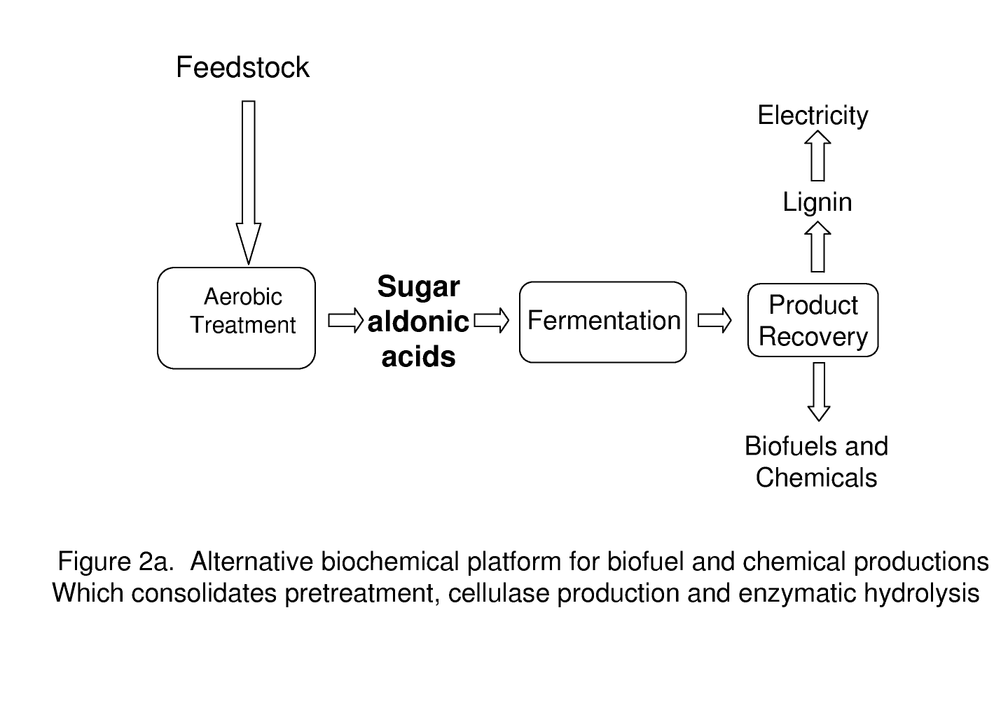

FIG. 2b depicts a special case of the biochemical platform disclosed herein for fuel- and chemical-productions by conversion of cellulosic biomass into sugar aldonic acids, which are then fermented to produce energy, biofuels, and chemicals, when a pre-treatment step is needed before the aerobic treatment.

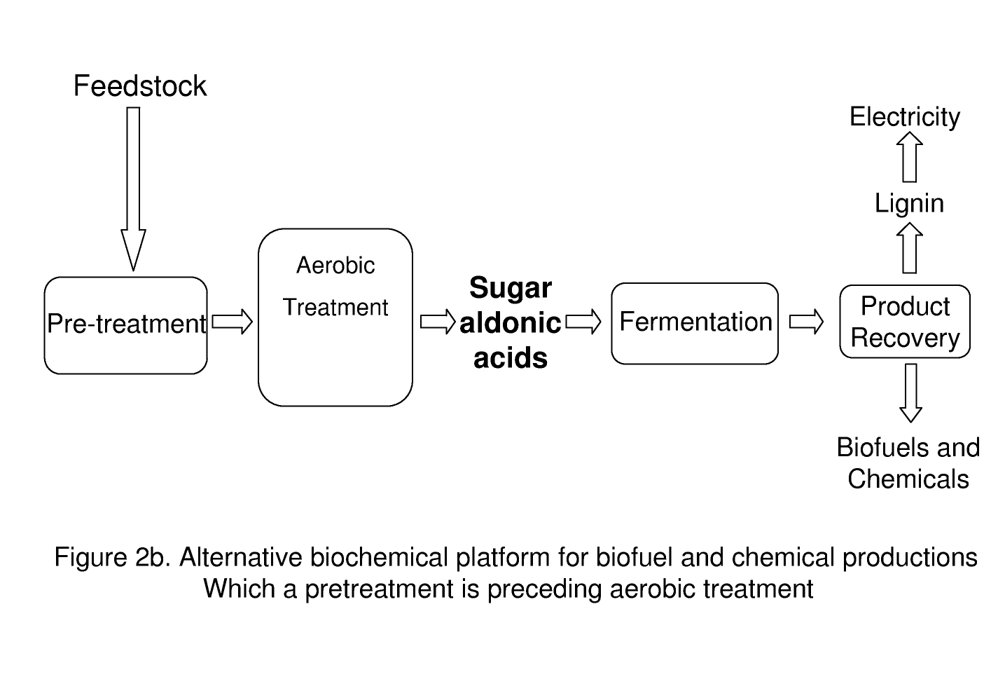

FIG. 3a is a diagram of cellulose hydrolysis and metabolism by certain cellulolytic microorganism(s).

FIG. 3b is a diagram of xylan hydrolysis and metabolism by certain cellulolytic microorganism(s).

FIG. 4 depicts a potential mechanism of cellulose degradation by White Rot Fungi (9).

FIG. 5 is a diagram of glucose flow after being hydrolyzed from cellulose.

FIG. 6 depicts the cellobionate production by wild type and quadruple bgl knockout strain XC-3456 of Neurospora crassa.

FIG. 7a depicts the production of cellobionate by wild type and seven bgl knockout WH-1234567 strains of N. crassa upon addition of exogenous cellobiose dehydrogenase.

FIG. 7b depicts cellobionate production from cellobiose upon addition of cellobiose dehydrogenase.

FIG. 8 depicts the conversion of gluconic acid to ethanol and acetic acid using Z. mobilis.

FIG. 9 depicts ethanol and acetic acid production from low concentrations of glucose and gluconate by E. coli KO11.

FIG. 10 depicts ethanol and acetic acid production from high concentrations of glucose and gluconate by E. coli KO11.

FIG. 11 depicts the conversion of gluconate to ethanol and acetic acid by E. coli KO11.

FIG. 12 depicts the co-fermentation of gluconate and glucose to ethanol and acetic acid by E. coli KO11.

FIG. 13 depicts the ethanol production from an equimolar mixture of glucose and gluconate by E. coli KO11.

FIG. 14 depicts the conversion of cellobionate to ethanol and gluconate by the yeast S. cerevisiae.

### DETAILED DESCRIPTION

The following description sets forth exemplary methods, parameters and the like. It should be recognized, however, that such description is not intended as a limitation on the scope of the present proposed invention but is instead provided as a description of exemplary embodiments.

### Overview

The methods, also referred to as production platforms, described herein allow for the production of fuels and chemicals from cellulosic biomass. The method has two main parts. The first part involves the conversion of carbohydrate contained in cellulosic biomass to sugar acids, specifically saccharide aldonic acids (SAAs) and their salts, via aerobic treatment. In the second part, said SAAs will be utilized as the reactive intermediates to produce ethanol or other chemicals by anaerobic fermentation.

The first part of the platform will utilize aerobic microorganism(s), which produce enzymes that hydrolyze cellulose and hemicellulose to resulting sugars while simultaneously degrading lignin, solubilizing lignin or changing lignin to a revised form, such as de-methylized lignin. Lignin is an energy-rich compound that can be utilized for energy production (e.g. electricity). In one embodiment, the majority of the sugars formed will be oxidized to corresponding sugar acids and their salts such as cellooligosccharide aldonic acid, cellobionic acid, gluconic acid, xylobionic acid, xylonic acid, mannonic acid, arabinic acids and galactonic acid, thus preventing sugar utilization by the microorganisms. A small fraction of sugar or sugar acids will be utilized to support cell growth and enzyme production.

After the initial step of sugar acid production, the second part of the process involves an anaerobic or aerobic treatment step that will convert the SAAs via fermentation into chemicals including acetic acid and fuels such as ethanol, butanol, and other hydrocarbons. In another embodiment, the method further includes co-fermenting SAAs with other carbon sources to produce the desired fermentation product(s).

### Part I: Aerobic Treatment: Conversion of Carbohydrate Contained in Cellulosic Biomass to Saccharide Aldonic Acids (SAAs)

The population of microorganisms utilized by aerobic treatment for the conversion of cellulosic materials into SAAs may be comprised of a single species of microorganism or a consortium of microorganisms. These organisms may be naturally-occurring or genetically-modified. In one embodiment, the microorganism or consortium of microorganisms shall express one or more of the following enzymes: cellulases, xylanases, ligninases, oxidases (dehydrogenases), and laccases.

Saccharide aldonic acids (SAAs) refer to molecules in which the CHO aldehyde functional group of a saccharide has been replaced with a carboxylic acid functional group (COOH). They can be divided into four general categories: (1) oligosaccharide aldonic acid (OAA), (2) di-saccharide aldonic acid (DAA), (3) monosaccharide aldonic acid (MAA), and (4) heteropolysaccharide aldonic acid (HSAA). Examples of oligosaccharide aldonic acid (OAA) include: cellotrionic acid, cellotetraonic acid; celloheptonic acid, xylotrionic acid, and xylopentaonic acid. Examples of di-saccharide aldonic acid (DAA) include: cellobionic acid (CBA), xylobionic acid, galactonic acid, 4-O-β-D-galactopyranosylgluconic acid, and 6-O-β-D-galactopyranosylgluconic acid. Examples of monosaccharide aldonic acids (MAA) include gluconic acid, xylonic acid, galactonic acid, arabiononic acid, and mannonic acid. An example of HSAA is 4-O-methyl-α-D-glucuronopyranosyl acid.

In the case of DAA, OAA and HSAA, the connection between sugar units and between the sugar and the end of the aldonic acid could be straight-chain or branched-chain. For example, gluconic acids could be connected glycosidically on the oxygen atoms in the 1-, 3-, 4- or 6-position of a sugar unit. There could be any combination of sugar and terminal aldonic acid.

Examples of inorganic and organic salts are ammonium, lithium, sodium, magnesium, calcium and aluminum salts, as well as the salts with ethanolamine, triethanolamine, morpholine, pyridine, and piperidine.

### Preparation of Microorganism

The population of microorganisms used for aerobic treatment can be constructed by at least the following two options.

Option 1 involves blocking or reducing the expression level of β-glucosidase and/or cellobiase by gene knockout, gene knockdown or by the addition of chemical inhibitors that will lead to sugar acid production in the cellulolytic microorganism(s). Those microorganisms may include cellulolytic fungi, yeast, and bacteria. Cellulolytic fungi may include White Rot Fungi, Brown Rot Fungi, Soft Rot Fungi or ascomycetes fungi such as Neurospora, Aspergillus, and Trichoderma. Within this group, White Rot Fungi possess lignin degradation enzymes that can degrade cellulose and hemicellulose in addition to lignin. In contrast, Neurospora, Brown Rot Fungi, and Soft Rot Fungi can degrade cellulose and hemicellulose but can only modify lignin. By blocking β-glucosidase and/or cellobiase activity, aerobic treatment will lead to production of OSA, DSA, MSA, and their salts.

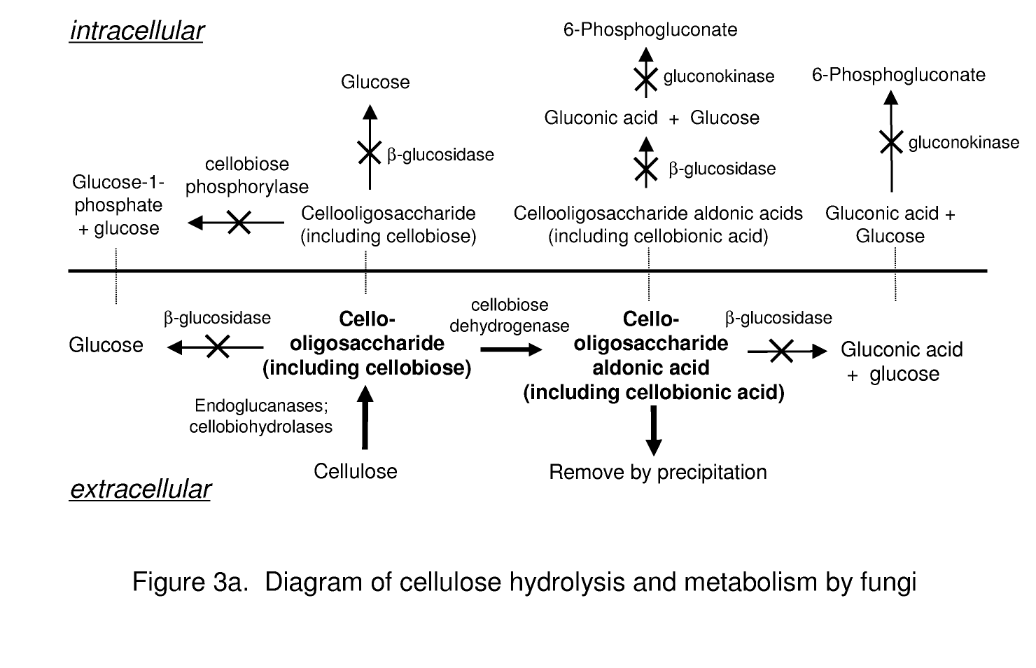

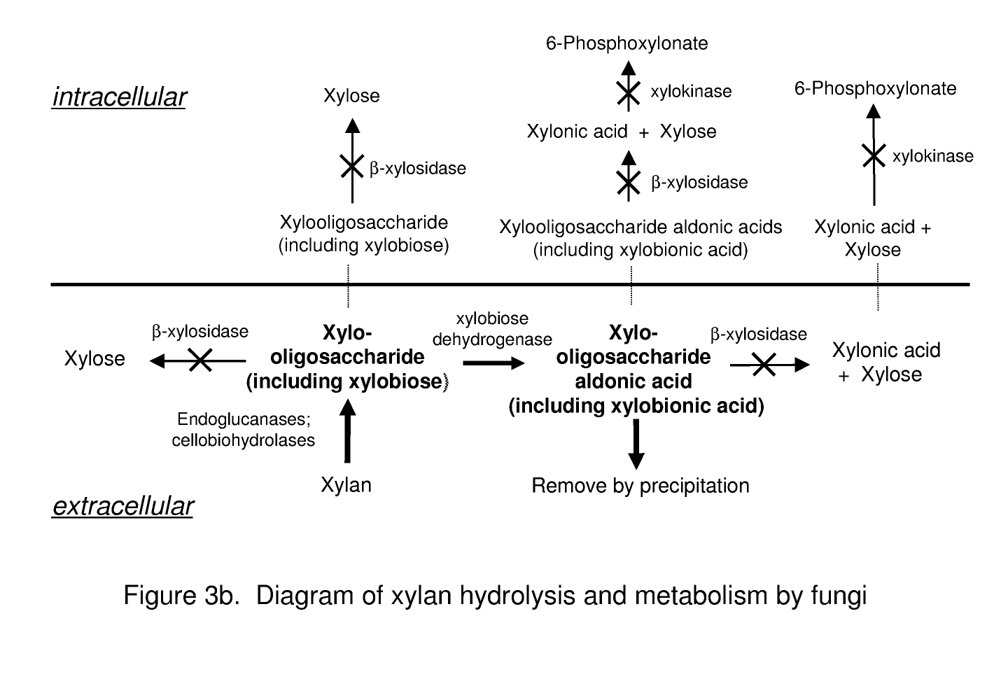

FIG. 3a diagrams the process of cellulose hydrolysis and metabolism by fungi. During hydrolysis of cellulose, the fungal endoglucanases and cellobiohydrolases will break the cellulose down into soluble cellooligosccharides and cellobiose. The enzyme cellobiose dehydrogenase (CDH) will then oxidize cellobiose to cellobionolactone, which reacts spontaneously with water to form cellobionic acid. Both cellobiose and cellobionic acid can be taken up by the fungi. They can be cleaved to form glucose, and glucose and gluconic acid, by intracellular and extracellular β-glucosidase respectively. Both glucose and gluconic acid can be metabolized by fungi. If the expression of β-glucosidase is blocked or lowered, cellobiose will be diverted to produce cellobionic acid, which could then be removed from the product-stream by precipitation. Removal of the product will prevent any inhibition of the enzyme that could occur through negative feedback. CDH and β-glucosidase can also utilize cellooligosaccharides as substrates. CDH can oxidize a cellooligosaccharide to the correspondent cellooligosaccharide aldonic acid (COAA) while β-glucosidase can hydrolyze COAA and cellooligosccharide to correspondent monomeric sugars and MSA. Hence, blocking β-glucosidase will lead to COAA production in addition to cellobionic acid production.

For cellulolytic microorganisms such as Trichoderma and Aspergillus, the strategy would need to be combined with the heterologous expression or exogenous addition of CDH and/or cellooligosaccharide oxidases.

For all cellulolytic microorganisms, the sugar acid yield from cellulose could be improved by increasing the expression level of CDH, cellooligosaccharide dehydrogenase (oxidases), and glucose oxidases either independently or in combination with blocking or inhibiting gluconate utilization. All these manipulations could be done simultaneously, in various combinations or independently.

As shown in FIG. 3b, a similar approach can be adopted for the conversion of hemicellulose (e.g. xylan) to sugar acids. Blocking or inhibiting β-xylosidase activity will lead to sugar acid production resulting from oxidation of the intermediate formed during hemicellulose hydrolysis. Such sugar acids include xylonic acid, xylobionic acid, xylooligosaccharide aldonic acid, galactonic acid, arabinonic acid, and HSAAs. Homologously or heterologously over-expressing xylobiose dehydrogenase (XDH), xylose oxidase (dehydrogenase), galactose oxidase, arabinose oxidase, and xylose oxidase, and/or blocking or inhibiting xylonate, mannonate, arabinonate, and galactonate utilization will increase the overall sugar acid yield and production rates. All these manipulations could be done simultaneously, in various combinations or independently.

The conversion of cellulose and hemicellulose portions of cellulosic biomass to the sugars described above could be conducted separately or simultaneously.

In all the methods mentioned above, improving the expression levels of cellulases, xylanases, ligninases, oxidases, and laccases individually, in combination or simultaneously may improve the rate and yield of sugar acid production.

In all the methods mentioned, the expression levels of the enzymes could be replaced or supplemented by exogenously adding that specific enzyme. The organisms may also be genetically modified to express recombinant enzymes at or above wild type levels

Option 2 involves developing an aerobic treatment process involving a microbial consortium that starts with a system comprising aerobic cellulolytic bacteria/yeast/fungi that produce cellulases and/or xylanases, to be combined with other aerobic microorganisms that can contribute either one or all of the enzymes needed including lignin peroxidase, CDH, cellooligosaccharide oxidases, laccases, glucose oxidases, xylose oxidases, and mannose oxidases. β-glucosidase and/or cellobiase activity is then blocked in the comprising strains. At least one of the strains should produce CDH or cellooligosaccharide oxidase and/or glucose oxidases that will lead to production of sugar acids.

A similar approach can be adopted for the conversion of hemicellulose (e.g. xylan) to sugar acids. Blocking or inhibiting β-xylosidase activity will lead to sugar acid production resulting from oxidation of the intermediate formed during hemicellulose hydrolysis. Such sugar acids include xylonic acid, xylobionic acid, xylooligosaccharide aldonic acid, galactonic acid, arabinonic acid, and HSAAs. Homologously or heterologously over-expressing XDH, xylose oxidase (dehydrogenase), galactose oxidase, arabinose oxidase, and xylose oxidase, and/or blocking or inhibiting xylonate, mannonate, arabinonate, and galactonate utilization will increase the overall sugar acid yield and production rates. All these manipulations could be done simultaneously, in various combinations or independently.

The conversion of cellulose and hemicellulose portions of cellulosic biomass to the sugars described above could be conducted separately or simultaneously.

In all the methods mentioned above, improving the expression levels of cellulases, xylanases, ligninases, oxidases, and laccases individually, in combination or simultaneously may improve the rate and yield of sugar acid production.

It is proposed that in all the methods mentioned above, blocking or lowering the expression level of cellobiose phosphorylase and/or xylobiose phosphorylase can lead to higher sugar acid production

It is proposed that in all the methods mentioned above, blocking or lowering the expression level of catabolite repression proteins, which down-regulate cellulase and other hydyolases production, can lead to higher sugar acid production

It is further proposed that in all the methods mentioned above, blocking or lowering the expression level of gluconokinase or xylonokinase can lead to higher sugar acid production.

In all the methods mentioned for option 2, the expression levels of the enzymes could be replaced or supplemented by exogenously adding that specific enzyme. The organisms may also be genetically modified to express recombinant enzymes at or above wild type levels.

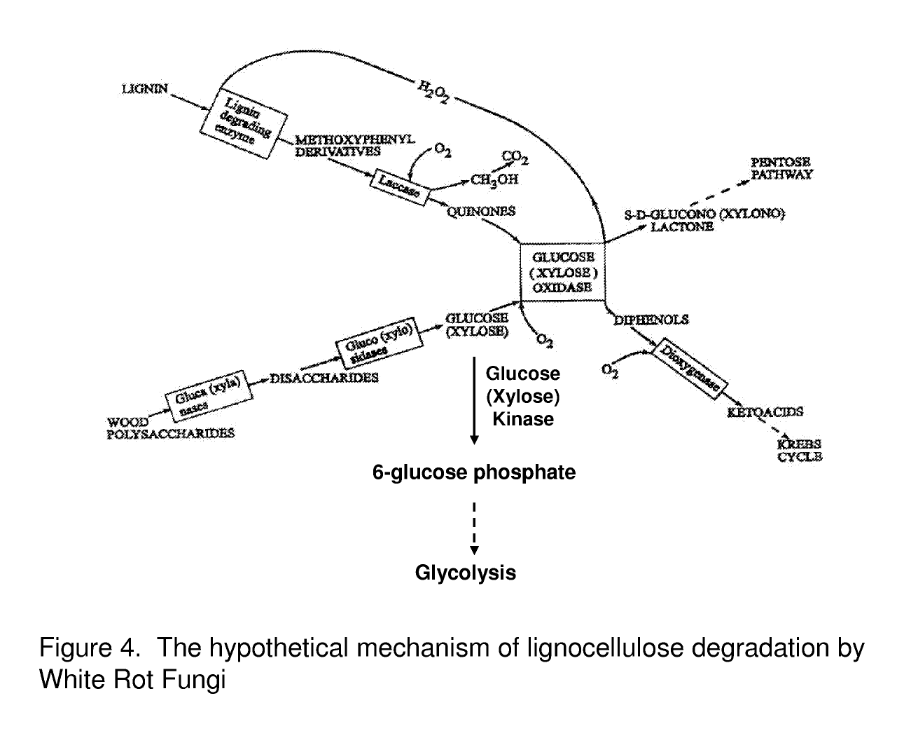

White Rot Fungi has been the focus of many studies as the agent for biological removal of lignin (10-13) and degradation of organic substances (14-16). It possesses all the enzymes needed to degrade components of cellulose (6-9). These enzymes include enzymes responsible for cellulose hydrolysis such as exoglucanases, endoglucanases and cellobiases, enzymes responsible for xylan hydrolysis such as endoxylanases and glucuroniases, and enzymes responsible for lignin degradation such as lignin peroxidases and manganese-dependent peroxidases. White Rot Fungi also secrete enzymes that do not directly interact with cellulose but can assist in carbohydrate hydrolysis and lignin degradation. Those enzymes include glucose oxidases (GOD), cellobiose oxidases, and laccases. FIG. 4 depicts a hypothetical mechanism of cellulose degradation by White Rot Fungi (9).

GOD is an important enzyme for cellulose degradation (17, 18). GOD is the primary source of peroxide (H₂O₂), which is necessary for lignin peroxidase activity (9, 17, 19, 20). Its importance in lignin degradation is demonstrated by the inability of a GOD-deficient mutant to degrade lignin (17). In addition, the oxidation of glucose reduces quinoid and phenoxy radicals yielded by laccase during the oxidation of lignin (21), which is also essential for lignin degradation. Glucose is the substrate of GOD and gluconate is the product of the GOD-catalyzed reaction. White Rot Fungi is able to metabolize both glucose and gluconate. However, they are metabolized via two different pathways. Glucose can be phosphorylated to form glucose-6-phosphate and enter either glycolysis or the pentose phosphate pathway. In contrast, the metabolism of gluconic acid involves phosphorylation by gluconokinase to 6-phosphogluconate, which is subsequently metabolized via the pentose phosphate pathway. Blocking gluconate utilization by the microorganism can be achieved by knocking out the gluconokinase gene, which will not affect glucose utilization. Glucose can then be irreversibly converted to gluconate by GOD, thereby preventing it from undergoing microbial utilization. Moreover, gluconate can be removed from the product stream by calcium carbonate (CaCO₃) precipitation or other methods to avoid any inhibition of the enzyme.

Over-expression of GOD is expected to accelerate lignin degradation since the GOD-catalyzed reaction involves two functions necessary for the acceleration of lignocellulose breakdown. Glucose can be converted to gluconate faster than it can be utilized by microbes. Initial calculation suggests that the rate of converting glucose to gluconate by GOD could be at least nine times faster than the glucose uptake rate by cells under a wide variety of glucose concentrations. The detailed estimations and calculation methods are described below.

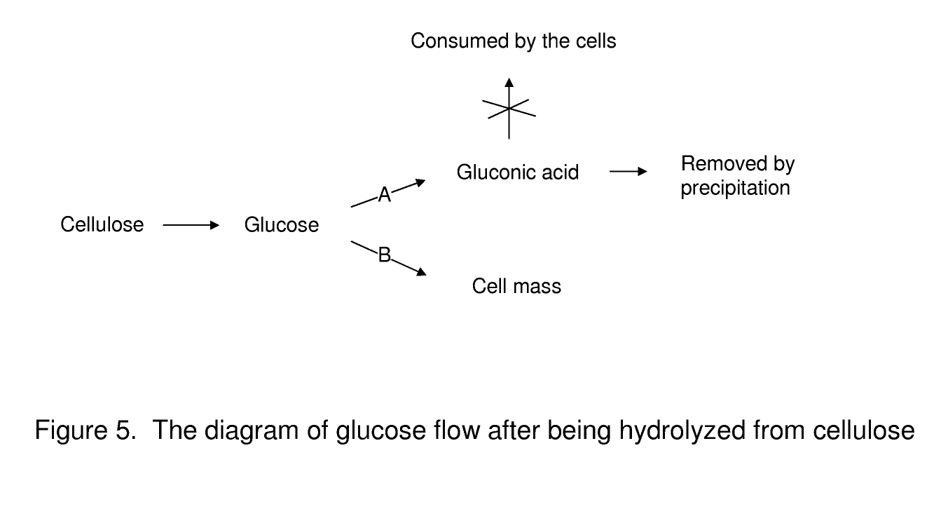

FIG. 5 diagrams the flow of glucose after being hydrolyzed from cellulose. The glucose will either be taken up by the cell to be converted to cell mass and to produce enzymes or it will be converted to gluconate. The flow rate in each route is dependent on the relative rates of reaction A (oxidation reaction) and B (glucose uptake). The reaction rate of oxidation is dependent on the GOD and glucose concentrations.

$$\frac{d G}{d t} = \frac{{k}_{cat} [ E ] [ G ]}{{k}_{m} + [ G ]}$$

Where:
   [E]: enzyme concentration
   Kₘ: Michaelis-Menten constant
   [G]: concentration of glucose in the broth
   T: time

In ethanol production by the traditional chemical platform, Wooley et al. has calculated that approximately 2% of the carbohydrate resulting from pre-treatment will be required to produce the enzymes needed to hydrolyze the remaining 98% of cellulose using Trichoderma as the cellulase producer, and that the protein yield from carbohydrate is about 0.3 g protein/g carbohydrate by the strains. Conservative estimates suggest that White Rot Fungi would need to devote about 10% of the carbohydrate for production of cell mass, cellulases, and other enzymes to hydrolyze the remaining 90% of carbohydrate. The protein yield from carbohydrate is 0.1 g/g carbohydrate. Estimations suggest that the strain can be engineered to ensure that GOD constitutes 1% of the total amount of synthesized protein. Estimations regarding White Rot Fungi are believed to be achievable process development goals, given how genetic engineering and strain selection have improved productions of cellulase and other extracellular enzymes. The improvement could conceivably occur via many mechanisms, including higher overall expression levels of enzymes, more optimal ratios of different cooperating enzymes activities, improved enzyme secretions, and improvement of specific activities of the enzymes.

TABLE 1
Parameters used in the calculation

| | Estimated Value | | |
|---|---|---|---|
| | used in | Reference | |
| | calculation | Point | Reference |
| % of carbohydrate devoted | 10% | 2% | Wooley et |
| for cell mass production | | | al., 1998 |
| Protein yield g/g | 0.1 | 0.3 | |
| carbohydrate | | | |
| Glucose oxidase percentage | 1% | N/A | |
| among protein | | | |
| The time needed to finish | 5 days | | |
| the fermentation | | | |
| k_{cat} of GOD | 1000 | 2001 S⁻¹ | (22) |
| K_{M} of GOD | 2 | 1.03 g/L | (22) |

It was further estimated that the strain is able to consume 10 g/L of glucose in five days and an average glucose consumption rate was calculated based on that value. The rate of glucose oxidation under several glucose concentrations was calculated. As shown below in Table 2, glucose oxidation rate would be at least nine times higher than its rate of uptake in very low glucose concentrations.

TABLE 2
Comparison of glucose uptake rate and oxidation rate:

| Assuming | Oxidation | | |
|---|---|---|---|
| glucose | rate | Glucose update | |
| concentration in | (A) | rate: (B) | Ratio of |
| the broth (mg/L) | (mg/L/S) | (mg/L/S) | A/B |
| 10 | 49.75 | 0.023 | 2149 |
| 1 | 5.00 | 0.023 | 215 |
| 0.1 | 0.50 | 0.023 | 21.5 |
| 0.01 | 0.05 | 0.023 | 2.15 |

### Part II: Anaerobic Treatment: Conversion of Sugar Acids to Fuels and Chemicals

In Part II of the method described herein, an anaerobic or aerobic fermentation step is employed for the conversion of SAAs and their salts to produce fuels and chemicals. The definition of SAAs and their salts is the same as in the previous section.

The conversion of COAA and CBA and their salts to ethanol may be achieved with the addition of an enzyme such as β-glucosidase, or via acid hydrolysis with the addition of an acid to break down COAA and CBA to glucose and gluconic acid, followed by fermentation of the glucose and gluconic acid aerobically or anaerobically, individually or together, to produce different fermentation products. Alternatively, strains of bacteria, such as E. coli, that naturally utilize COAA and CBA or their salts to produce ethanol can be utilized.

In an alternative method, a microorganism may be genetically modified to utilize COAA, CBA, and their salts. For example, Z. mobilis may be engineered with β-glucosidase so it can break down COAA and CBA intracellularly and then convert gluconic acid to ethanol.

Gluconic acid and its salt are very important industrial chemicals that have wide applications in pharmaceutical, food, textile, detergent, leather and other biological industries (23, 24). It has also been identified as one of the less-than-20 primary building blocks for future bio-refinement since a wide variety of chemicals can be derived from it. Moreover, gluconic acid is a readily available carbon source for microbial fermentation. Multiple products can be derived from gluconate fermentation including ethanol. For example, gluconic acid can be metabolized via 6-phosphogluconate to 2-keto-3-deoxy-6-phosphogluconate and enter the Entner-Doudoroff pathway and eventually be converted to ethanol by Z. mobilis or E. coli. Z. mobilis possesses all the enzymes needed to ferment gluconate to ethanol (25). Typically, gluconate cannot be metabolized by the yeast Saccharomyces cerevisiae directly because the organism lacks the gluconate kinase necessary to phosphorylate gluconate. However, this deficiency may be addressed by transforming a recombinant gluconic acid kinase gene into S. cerevisiae. Once S. cerevisiae expressed the gluconic acid kinase, gluconate can be metabolized to 6-phosphogluconate and enter the pentose phosphate pathway to form various fermentation products.

The overall reaction to convert gluconic acid to ethanol, and for CBA to ethanol, could be described using the following equations:

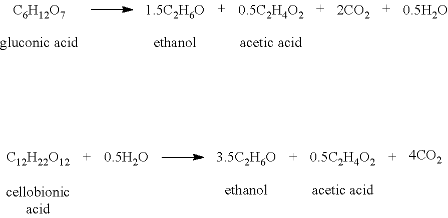

One mole of gluconic acid could yield 1.5 mole of ethanol and 0.5 mole of acetic acid. It produces 0.5 mole less ethanol than when glucose as the substrate. However, it produces 0.5 mole of acetic acid, which is an important industrial chemical. The concentration of ethanol produced during the process could conceivably be higher than that produced by the conventional simultaneous saccharification and fermentation (SSF) process. In the conventional SSF process, the ethanol concentration cannot exceed 5% (12) as ethanol has an inhibitory effect on the cellulase enzymes. Since the anaerobic fermentation process involves no enzymes, ethanol and acetic acid could be produced at higher concentrations, which will reduce the downstream product recovery costs.

The method also includes utilization of the sugar acid to be co-fermented with another carbon source to produce the desired fermentation product. Theoretically, gluconic acid as well as CBA could each be co-fermented with another substrate, which is more reduced than glucose such as glycerol and which is an abundant biodiesel industry by-product, to provide the reduction power balance. Examples of co-fermentation include the co-fermentation of gluconic acid or gluconate with glycerol to produce ethanol and the co-fermentation of CBA with glycerol for the production of ethanol. The overall reaction for co-fermenting gluconic acid and glycerol for ethanol production would be:

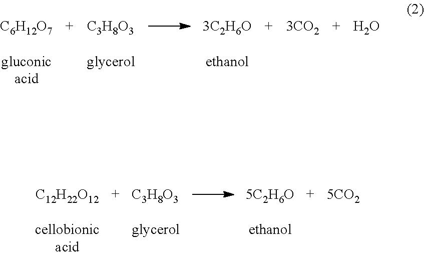

The overall reaction for co-fermenting gluconic acid and hydrogen for ethanol production would be:

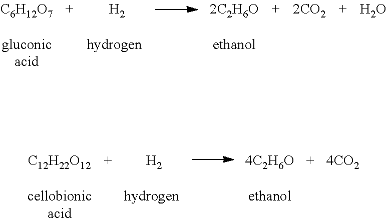

These methods may be more readily understood through the following non-limiting examples.

### Example 1

### Aerobic Production of Cellobionate in Neurospora crassa

### Background

The metabolic engineering of lignocellulolytic microorganisms to produce sugar aldonates was tested by using the microorganism Neurospora crassa (N. crassa). N. crassa was chosen for its ability to produce all the required enzymes for the production of cellooligosaccharides, including cellobionate, from cellulosic biomass. N. crassa is a fast-growing fungus that produces a variety of oxidases and laccases that are involved in phenol degradation, and potentially in lignin modification. N. crassa can grow on un-pretreated wheat straw as a sole carbon source. Furthermore, N. crassa is a genetically tractable microorganism that has a sequenced genome. Tools for the genetic manipulation of N. crassa are readily available.

N. crassa expresses multiple β-glucosidase enzymes that may be involved in the metabolism of cellooligosaccharides by converting them to glucose or gluconate (FIG. 3a). There are seven N. crassa β-glucosidase genes (bgl) that are of interest. The seven bgl genes have the following accession numbers: NCU00130, NCU04952, NCU05577, NCU07487, NCU08054, NCU08755, and NCU03641. All seven of the N. crassa bgl genes have been knocked out and the single knockout strains are publicly available from the Fungal Genetics Stock Center (Kansas City, Mo.).

In the present study, an N. crassa strain XC-3456 was designed, which has bgl genes NCU05577, NCU07487, NCU08054, and NCU08755 knocked out. This study shows that strain XC-3456 produces more cellobionate than a wild-type N. crassa strain that is not missing any β-glucosidase genes. A triple bgl knockout strain XC-267, which has bgl genes NCU08755, NCU03641, and NCU04952 knocked out, was also used in this study to show that over-expression of CDH further enhances cellobionate production.

Construction of Quadruple β-Glucosidase Knockout N. crassa Strain XC-3456

Standard genetic crossing protocols were used to generate multiple knockout strains. Double bgl knockout strains were constructed following a standard mating protocol. Gene knockout strains were grown on solid Vogel's minimal medium at 25° C. for seven days. Conidia were then harvested and added to another gene knockout strain carrying an opposite mating type grown on the synthetic cross medium. Within a month, thousands of ascospores resulting from a compatible mating were recovered.

Asexual conidia were excluded from ascospores by treatment at 60° C. Double knockout strains were selected using a PCR-genotyping method as follows. Each single knockout mutant strain, obtained from the Fungal Genetics Stock Center (Kansas City, Mo.), contained a hygromycin resistance gene (hph^{r}) inserted within a specific β-glucosidase gene. Primers were designed based on the hph^{r} open reading frame and the flanking sequence of the knockout loci. The double knockout strains produced two PCR products corresponding to the two replaced genes. Two double knockout strains were created: strain XC-34, which contained knockouts of bgl genes NCU05577 and NCU07487 and strain XC-56, which contained knockouts of bgl genes NCU08054 and NCU08755. XC-34 and XC-56 were crossed, following the same procedures, to generate the quadruple knockout strain XC-3456.

Construction of Seven bgl Knockout N. crassa Strain WH-1234567

Standard genetic crossing protocols were used to generate multiple knockout strains as described above. The triple knockout strain XC-267 was constructed by crossing a double knockout strain XC-67 (NCU08755, NCU03641) with another single knockout strain NCU04952. The quadruple knockout strain XC-1345 was constructed by crossing a double knockout strain XC-34, which contains knockouts of NCU05577, NCU07487 and XC-17, which contains knockouts of bgl genes NCU00130 and NCU03641. The quadruple knockout XC-1345 was constructed by double cross X15 and X34. The seven knockout strain WH-1234567 was constructed by crossing XC-267 with XC-1345. WH-1234567 contains knockouts of NCU00130, NCU04952, NCU05577, NCU07487, NCU08054, NCU08755, and NCU03641.

### Production of Cellobionate in XC-3456

N. crassa strain XC-3456 was used to convert rice straw to cellobionate. XC-3456 and wild type N. crassa were inoculated into flasks containing 20 g/L rice straw in Vogel's minimal medium. When rice straw was incubated with XC-3456 for 48 hours, about 0.14 g/L cellobionate was produced. When rice straw was incubated with XC-3456 for 72 hours, 0.18 g/L cellobionate was produced. No cellobionate production was detected when wild-type N. crassa, in which none of the bgl genes are knocked out, was incubated with rice straw.

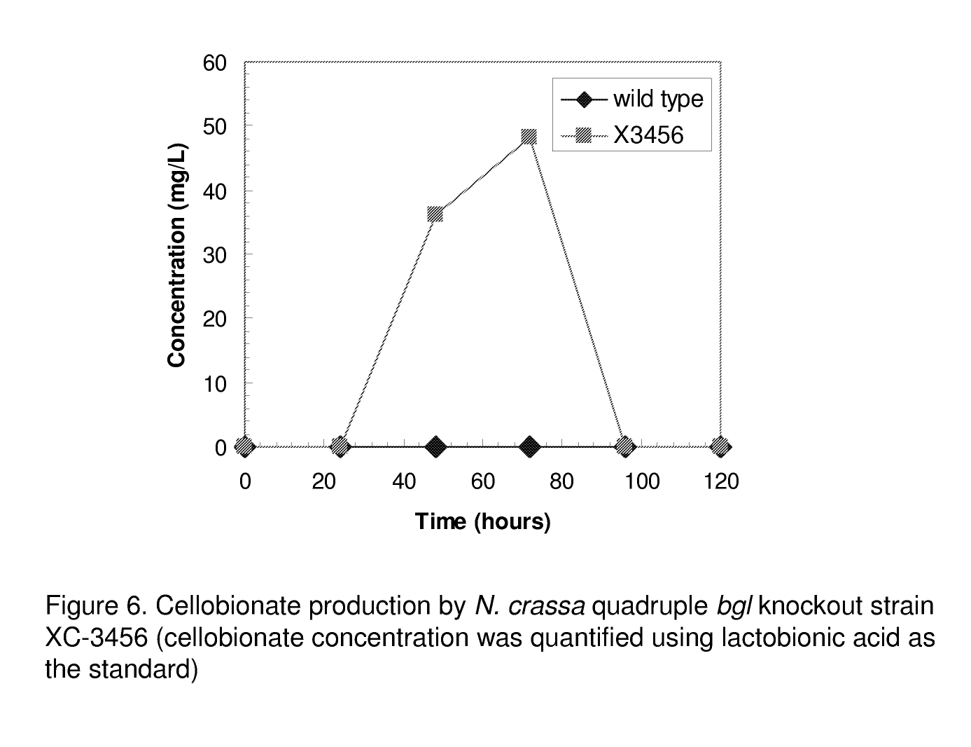

As shown in FIG. 6, N. crassa strain XC-3456 produces a maximum of 0.05 g/L of cellobionate (using Lactobionic acid as the standard) after about 70 hours. This result is in sharp contrast to that observed with wild type N. crassa, which produces no detectable levels of cellobionate at any time during the experimental time frame. The double bgl mutant strains XC-34 and XC-56 do not produce any detectable levels of cellobionate when compared to XC-3456. It is believed that the cellobionate produced by the XC-3456 was consumed after about 100 hours by the N. crassa cells since N. crassa was still producing β-glucosidase. It is hypothesized that with more copies of bgl genes knocked out, the strain will produce more and consume less cellobionate.

### Production of Cellobiose Dehydrogenase

The recombinant cellobiose dehydrogenase is produced using the method described below.

### Strains

N. crassa FGSC 2489 was from Fungal Genetics Stock Center (University of Missouri, Kansas City, Mo., USA). P. pastoris strain X33 and the vector pPICZα B were purchased from Invitrogen (Carlsbad, Calif., USA). N. crassa strain FGSC 2489 was maintained on Vogel's medium supplied with 2% sucrose (30). P. pastoris strain X33 was maintained on YPD (10 g/L yeast extract, 20 g/l peptone and 10 g/L glucose) plates. All the transformants derived from X33 were maintained on YDP plates containing 100 μg/ml Zeocin.

### DNA Manipulations, Enzymes, and Sequence Analysis.

DNA manipulations were performed using standard techniques (31). Plasmid was prepared using Miniprep kit (Qiagen, Valencia, Calif., USA). Gel extraction and PCR product purification were conducted using Qiaquick gel extraction kit and Qiaquick PCR purification kit respectively (Qiagen, Valencia, Calif., USA). All the restriction endonucleases, the Finnzyme Phusion-High-Fidelity DNA Polymerase, and T4 DNA ligase were purchased from New England Biolabs (Ipswich, Mass., USA). Oligonucleotide primers were synthesized by Invitrogen. DNA sequences were determined in DNA sequencing laboratory in University of California at Davis. Sequence analysis and alignments were conducted using Vector NTI software (Invitrogen, Carlsbad, Calif.).

Cloning of the cdh Gene cDNA Fragment

N. crassa strain FGSC 2489 was grown for 5 days at 30° C. on Vogel's plate supplied with 2% rice straw as carbon source. The mycelia were collected from the plates and shock-frozen in liquid nitrogen. Total RNA was isolated using the RNeasy Plant Mini System (Qiagen, Valencia, Calif., USA). The first strand cDNA was obtained with the ThermoScript RT-PCR System from Invitrogen using oligo (dT)₂₀ primers. Then, cDNA fragments were amplified using forward primer (5′-ATTACTGCAGGACAAACCGCTCCCAAG-3′, the introduced Pst I site was underlined) and reverse primer (5′-AGTCTCTAGACCCACACACTGCCAATA-3′, the introduced Xba I site was underlined) Finnzyme Phusion-High-Fidelity DNA Polymerase was used in the amplification. The predicted signal peptide sequence was not present in the amplified cDNA fragment.

### Construction of Expression Vectors

The NC-cdh cDNA was digested by Pst I and Xba I, and ligated into the Pst I/Xba I site of the vector pPICZα B, obtaining pPICZα B-cdh. The NC-cdh gene was downstream to the alcohol oxidase I (AOX I) promoter and the α-factor signal sequence. On the C-terminus, the NC-cdh gene was fused with the c-myc epitope and a His₆ tag sequence, encoded by pPICZα B. The insert was sequenced using AOX I sequencing primers and the NC-cdh internal primers and the cDNA sequence was confirmed correct.

### Transformation and Screening

P. pastoris X33 competent cells were prepared following the procedures as described in the Easy Select Pichia Expression System manual from Invitrogen. pPICZα B-cdh was linearized using Pme I. An Eppendorf electroporator 2510 (Eppendorf, Westbury, N.Y., USA) was utilized in the electroporation. Transformants were selected on YPD plates containing 100 and 1000 μg/ml Zeocin respectively.

### Heterologous Expression of Recombinant NC-cdh

Selected colonies were grown in YPG medium (10 g/L yeast extract, 20 g/l peptone and 10 g/L glycerol) in a rotary shaker (250 rpm) at 30° C. The cells were harvest by centrifugation (5 mM, 2500 g), and re-suspended in the same volume of YPM medium (10 g/L yeast extract, 20 g/L peptone and 10 ml/L methanol), incubated at 30° C., 250 rpm. Methanol was added at 1% concentration every 24 hours during the period of induction. Samples were taken at various time intervals. The cell density was monitored during the growing process. The protein concentration in the supernatants were determined using Bradford method (32). The CDH activity was also monitored using the enzyme assay method described below.

### Purification of the Recombinant CDH

200 ml culture supernatant was concentrated 20-fold using a PM-30 ultrafiltration membrane (Millipore, Bedford, Mass., USA). The concentrate was then applied to the Ni-NTA Purification System (Invitrogen, Carlsbad, Calif., USA) under native conditions. Ten milliliter concentrated solution was loaded onto a 10 ml Ni-NTA agarose resin, which had been equilibrated with the native binding buffer (50 mM NaH₂PO₄, 0.5 M NaCl, pH 8.0). Eight milliliter of native wash buffer (50 mM NaH₂PO₄, 0.5 M NaCl, 20 mM imidazole, pH 8.0) was applied for three times to wash the column. Then, the His₆-tagged NC-CDH was eluted from the column with 10 ml native elution buffer (50 mM NaH₂PO₄, 0.5 M NaCl, 250 mM imidazole, pH 8.0). The purified recombinant NC-CDH was dialyzed against 100 mM sodium acetate buffer (pH 4.5), concentrated to 10 ml and stored at 4° C. Protein concentration and CDH activity were subsequently determined

### Enzyme Assays and Cellobionate Measurement

Cellobiose dehydrogenase activity was measured by the reduction of 2,6-dichlorophenolindophenol (DCPIP) at 515 nm (e=6.8 mM⁻¹ cm⁻¹). The assay mixture contained 3 mM cellobiose, 100 mM sodium acetate (pH 4.5), 100 μM DCPIP, and various amounts of culture supernatant or purified protein to a total of 1 mL. If not pointed out, all the assays were performed at 30° C. Enzyme activity, expressed in international units (IU), was equivalent to the reduction of 1 mmol of DCPIP per minute. 1 μmol reduction of DCPIP is equivalent to 1 μmol of cellobionate generated.

Production of Cellobiose Using WH-1234567 with Exogenous Cellobiose Dehydrogenase Addition.

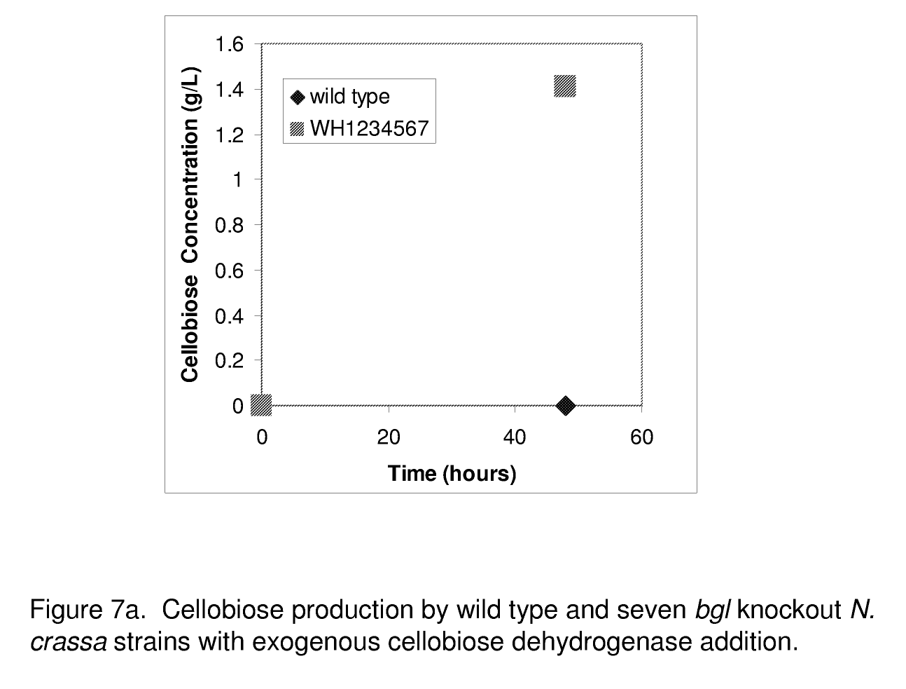

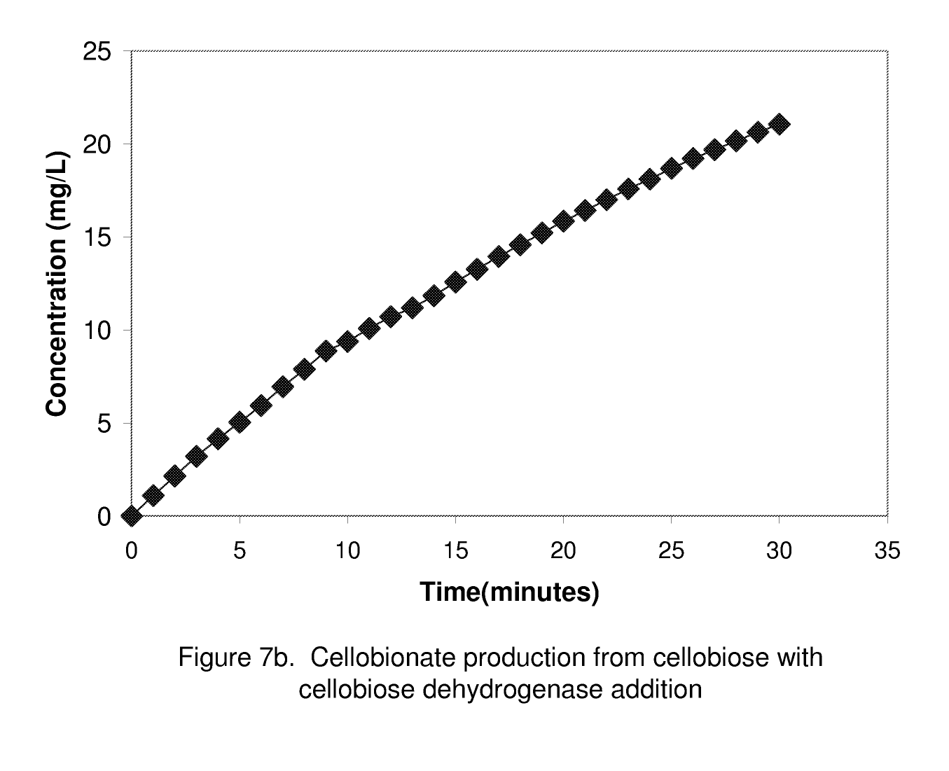

N. crassa strain WH-1234567 was used to convert rice straw to cellobionate. WH-1234567 and wild type N. crassa were inoculated into flasks containing 20 g/L rice straw in Vogel's minimal medium. The fermentation broth was collected at 48 hours and 1000 U cellobiose was added to the broth, after 24 hours, about 1.4 g/L cellobiose was detected in the fermentation broth produced by WH-1234567, no cellobiose was detected in the fermentation broth of wild type, as shown in FIG. 7a. When about 40 units of recombinant cellobiose dehydrogenase was added to 1.06 g/L cellobiose with 100 mM of DCPIP addition, as shown in FIG. 7b, more than 20 mg/L cellobionate was produced in 30 minutes.

As shown in FIG. 7a, wild-type N. crassa strain produces no detectable cellobiose upon addition of exogenous CDH even after about 50 hours. In contrast, addition of CDH to the seven bgl knockout strain WH-1234567 increases the cellobiose production to 1.4 g/L within the same time frame, which has shown to be able to convert to cellobionate via CDH addition. Therefore, exogenous addition of CDH to a seven bgl knock-out strain increases cellobionate production considerably. FIG. 7b shows cellobionate production from cellobiose upon addition of CDH.

### Example 2

### Conversion of Gluconic Acid to Ethanol and Acetic Acid Using Z. mobilis

The strain Z. mobilis (ATCC 29191) was utilized to produce ethanol from gluconate. Batch cultures were grown in 150 mL sealed serum bottles with a 100 mL working volume under an N₂ atmosphere. Both sodium gluconate and calcium gluconate were utilized as the carbon source in the test and synthetic media (26). Inoculums were prepared by picking a single growing colony from a plate and culturing it aerobically for 24 hours. A serum bottle containing 100 mL of media was inoculated with 3 mL of the resulting culture, and the serum bottle was incubated under anaerobic condition for 20 hours. 20-hour seed culture was then inoculated into serum bottles with the cell concentration standardized to 0.2 g/L. Samples were then taken at different time intervals.

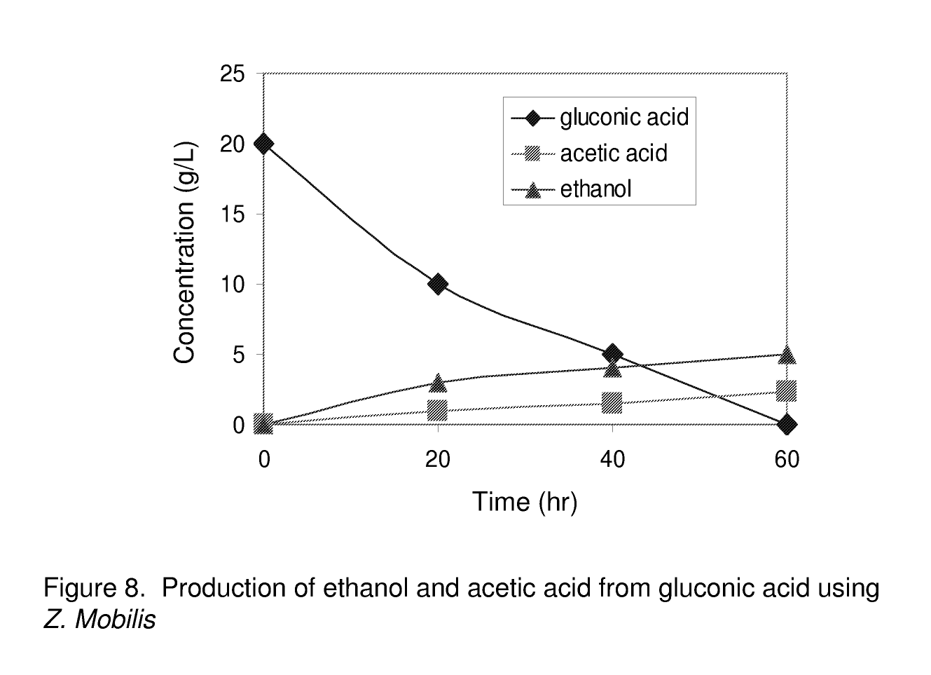

As shown in FIG. 8, starting with 20 g/L gluconate, Z. mobilis produces 5 g/L of ethanol (yield 25%), and 3 g/L of acetic acid (yield 15%). All of the gluconate is utilized within 60 hours. The pH does not change much during the course of fermentation.

### Example 3

### Conversion of Gluconate Using E. coli KO11

The E. coli strain KO11 was utilized to evaluate ethanol production from gluconate. The strain was maintained on agar plates containing 10 g/L glucose, 5 g/L yeast extract, and 10 g/L trypton. The inoculum was prepared by picking a single colony from the agar plate and culturing it aerobically for 24 hours using the medium mentioned above. The resulting culture was centrifuged at 4000 g to spin down the cells. Cells were then collected and inoculated into fermentation bottles with the cell concentration standardized to 0.2 g/L. The batch fermentation was carried out in 150 mL serum bottles with a working volume of 150 mL, using the same medium containing glucose, sodium gluconate, or calcium gluconate as the carbon source.

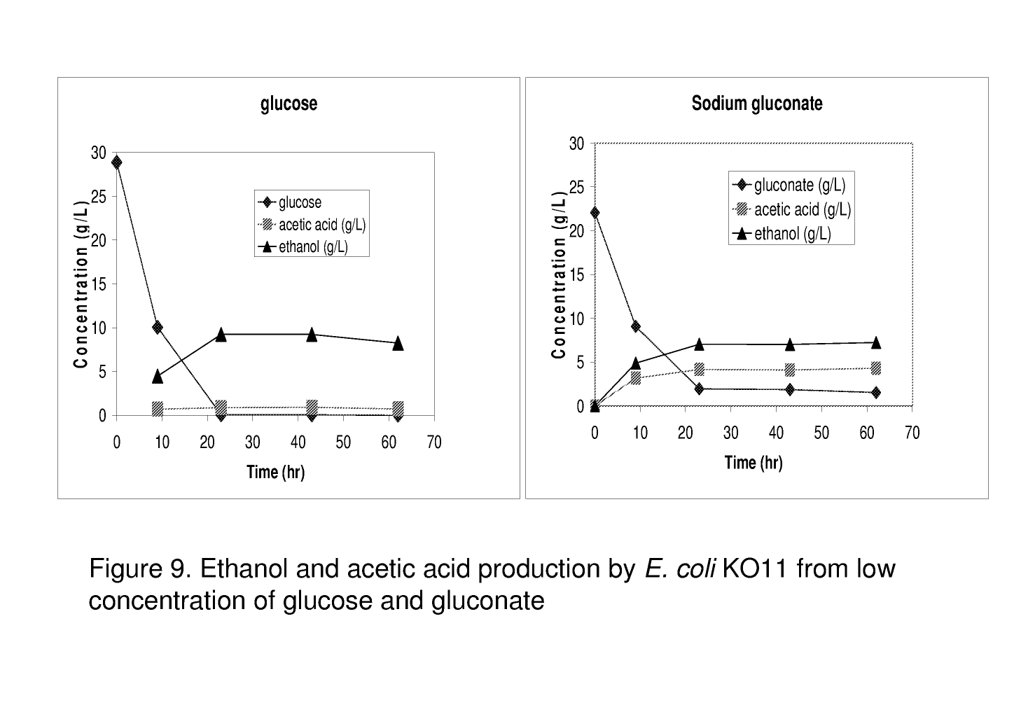

As shown in FIG. 9, starting with 28.8 g/L glucose, produces 9.2 g/L of ethanol (yield 31.9%, theoretical yield should be 51%). Starting with 22.g/L gluconate equivalent produced 7.04 g/L of ethanol (yield 32.8%, theoretical yield from equation 1 is 35%), and 4.3 g/L of acetic acid (yield 19.5%, theoretical yield from equation 1 is 15.3%) (FIG. 9). The pH of the culture at the start and end of the fermentation does not change. The rate of gluconate utilization is similar to that of glucose utilization.

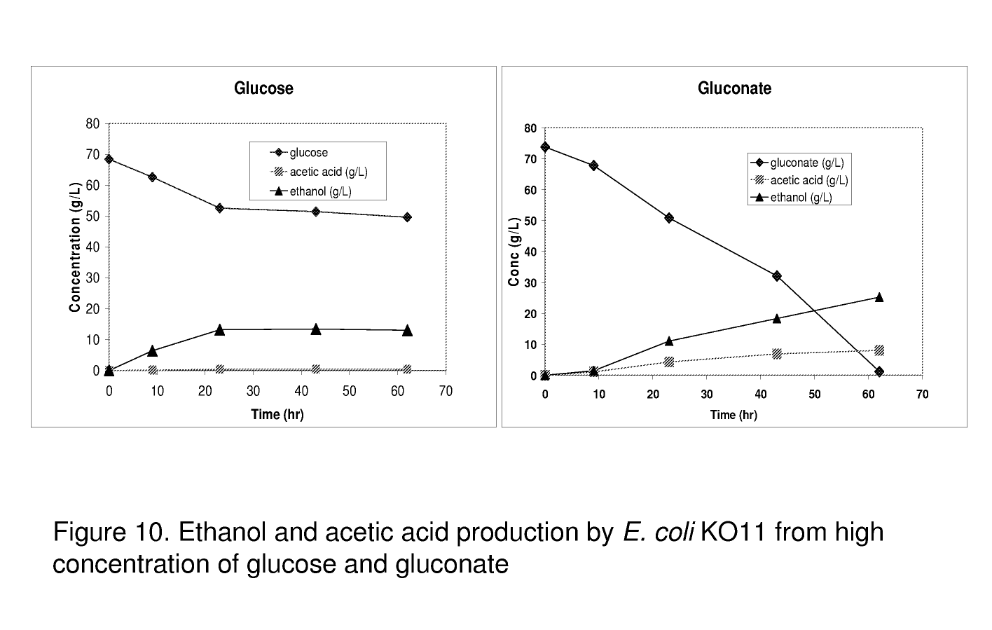

As shown in FIG. 10, increasing the starting glucose and gluconate concentration to around 70 g/L leads of a reduction in the fermentation rate of glucose. Only about 20 g/L of glucose is utilized during 62 hours. However, it was determined that the pH of the fermentation broth drops from a starting pH of 6.0 to an ending pH of around 4.4. This reduction in pH may have contributed to the slower fermentation of glucose. However, the fermentation did not decrease when gluconate was used as the carbon source. The gluconate is consumed within 62 hours, and about 25.3 g/L of ethanol is produced from 73.8 g/L gluconate (yield is 34.2%, theoretical yield from equation 1 is 35%), and 8.06 g/L of acetate is produced (yield is 10.9% theoretical yield is around 15.3%) (FIG. 10). The pH of the culture at the start and end of the fermentation did not change significantly (starting pH 6.0, ending pH 6.2).

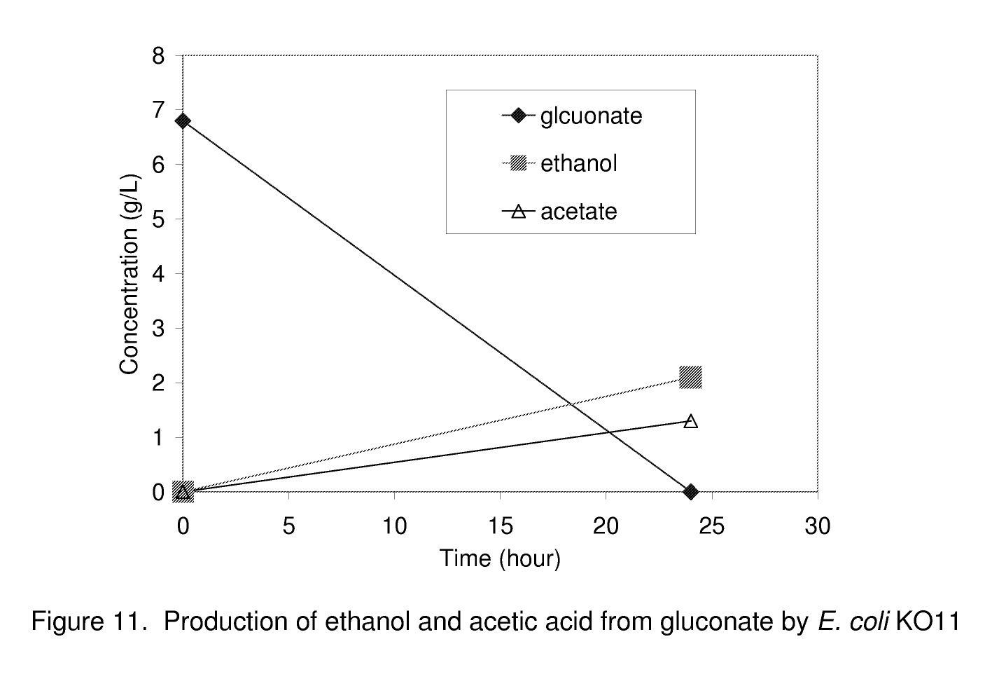

As shown in FIG. 11, starting with 6.9 g/L gluconate produces 2.4 g/L of ethanol (34.8% yield) and 1.3 g/L of acetic acid (18.8% yield). All of the gluconate is utilized within 25 hours. The pH of the culture at the beginning and end of the fermentation did not change significantly.

These experiments demonstrate that gluconate can be utilized as the carbon source for fuels and chemicals production.

### Example 4

### Co-Fermentation of Gluconate and Glucose Using E. Coli KO11

In the following experiments gluconate was mixed with glucose and fermented utilizing the E. coli strain KO11. The same procedure used in Example 3 was used in the following experiments.

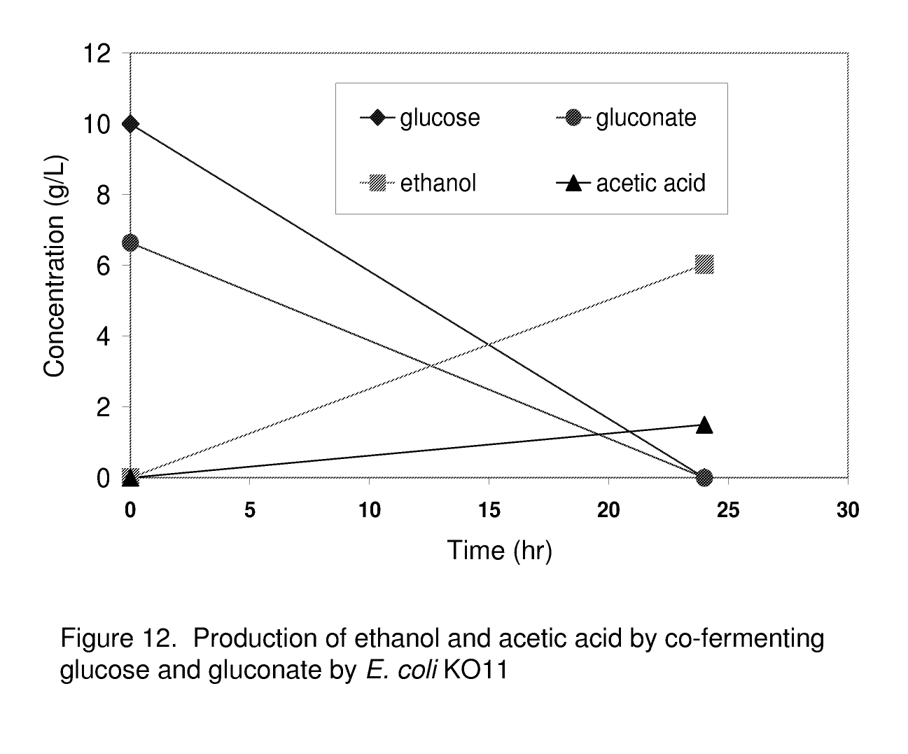

Starting with 6.2 g/L gluconate and 10 g/L glucose produces 6 g/L of ethanol and 1 g/L of acetic acid (FIG. 12). Both gluconate and glucose seem to be utilized at the same rate, and both are completely utilized by 25 hours (FIG. 12).

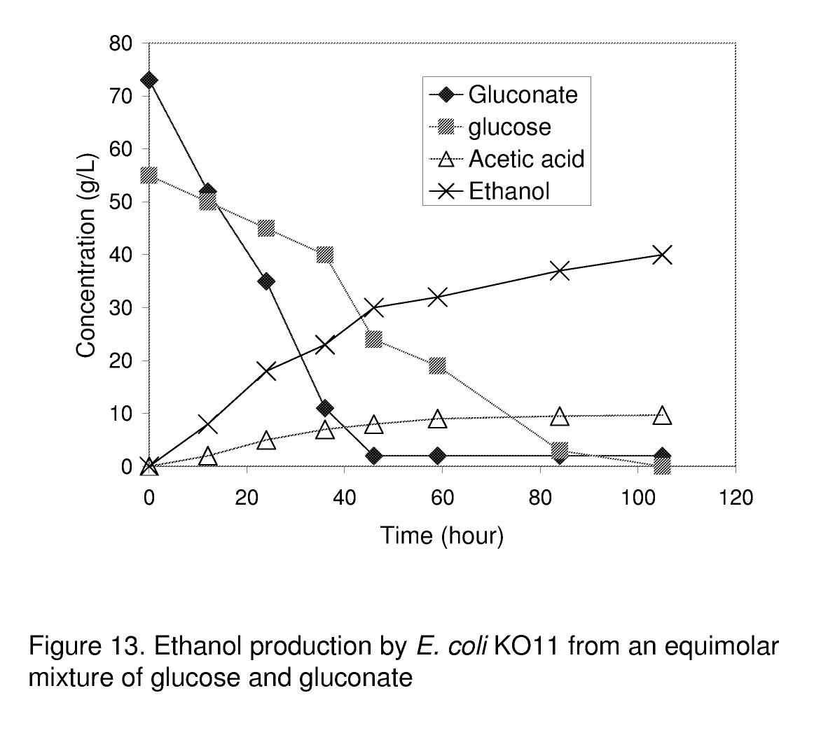

When an equal molar ratio mixture of gluconate and glucose are used, 40 g/L of ethanol is produced and 8 g/L of acetic acid is produced (FIG. 13). With higher starting concentrations of gluconate (65 g/L) and glucose (55 g/L), the rate of gluconate utilization seems to be higher than that for glucose. Despite having a higher starting concentration, all of the gluconate is utilized by 50 hours; however, glucose takes more than twice as long to be completely utilized (FIG. 13).

### Example 5

### Conversion of Cellobionate or Other Celloligosaccharide to Ethanol and Gluconate Using Saccharomyces cerevisiae

The strain S. cerevisiae was utilized to evaluate ethanol and gluconate production from cellooligosaccharide aldonates using cellobionate as an example. The strain was maintained on agar plate containing per liter: 10 g glucose, 10 g yeast extract, and 20 g peptone (pH 4.5). The batch fermentation was carried out in 80 mL serum bottles with a working volume of 30 mL using the same medium containing cellobionate (30 g/L) as the carbon source. β-glucosidase (Novozyme 188) was added at time zero at a loading of 200 IU/g cellobionate. The seed culture was prepared by culturing S. cerevisiae under anaerobic conditions using the medium mentioned above. One milliliter of 16-hour-old seed culture was inoculated into the fermentation serum bottle at time 12 hours. Samples were taken out at various time intervals. The concentration of cellobionate, gluconate, and ethanol were analyzed using a Biorad HPX-87H column following the standard protocol.

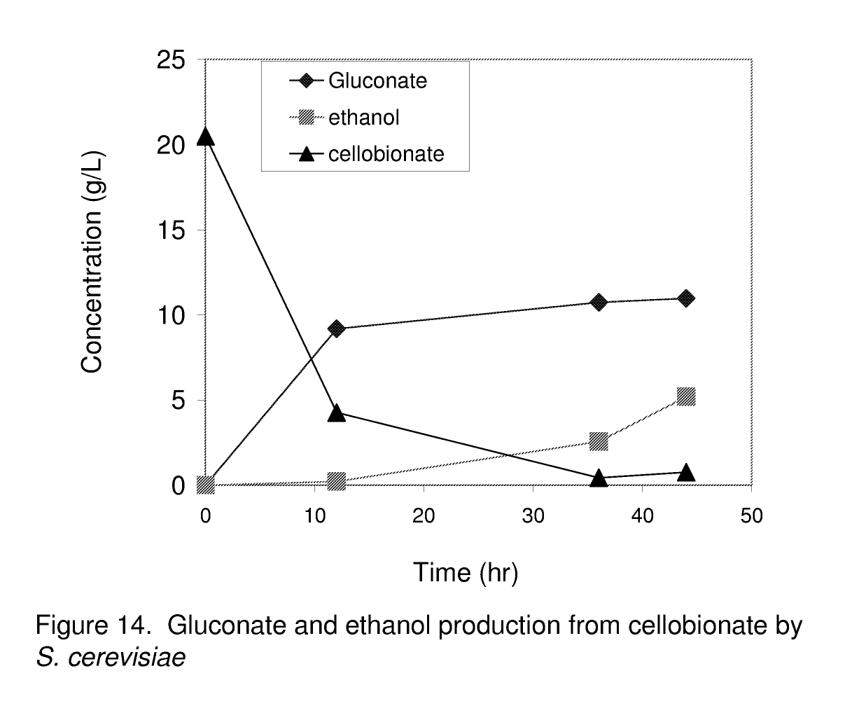

As shown in FIG. 14, S. cerevisiae is able to produce high concentrations of gluconate and ethanol from cellobionate. For instance, after about 45 minutes, gluconate and ethanol are produced at concentrations of about 10.5 and 5 g/L respectively. The concentration of cellobionate substrate falls from 20 g/L to about 0.7 g/L.

### REFERENCES

1. Lynd, L. R., Zyl, W. H., McBride, J. E., and Laser, M. (2005) Consolidated bioprocessing of cellulosic biomass: an update, Curr Opin Biotechnol.
2. Lynd, L. R., Weimer, P. J., van Zyl, W. H., and Pretorius, I. S. (2002) Microbial cellulose utilization: fundamentals and biotechnology, Microbiol Mol Biol Rev 66, 506-577, table of contents.
3. Lynd, L. R., Wyman, C. E., and Gerngross, T. U. (1999) Biocommodity Engineering, Biotechnol Prog 15, 777-793.
4. Lynd, L. R., Cushman, J. H., Nichols, R. J., and Wyman, C. E. (1991) Fuel Ethanol from Cellulosic Biomass, Science 251, 1318-1323.
5. Wooley, R., Ruth, M., Sheehan, J., Ibsen, K., Majdeski, H., and Galvez, A. (1998) Lignocellulosic biomass to ethanol process design and economics utilizing co-current dilute acid prehydrolysis and enzymatic hydrolysis current and futuristic scenarios, (NREL/TP-580-26157, N. R. E. L., Ed.).
6. Crawford, R. L., and Crawford, D. L. (1984) Recent Advances in Studies of the Mechanisms of Microbial-Degradation of Lignins, Enzyme Microb Technol 6, 434-442.
7. Kirk, T. K., Schultz, E., Connors, W. J., Lorenz, L. F., and Zeikus, J. G. (1978) Influence of Culture Parameters on Lignin Metabolism by Phanerochaete-Chrysosporium, Arch Microbiol 117, 277-285.
8. Worrall, J. J., Anagnost, S. E., and Zabel, R. A. (1997) Comparison of wood decay among diverse lignicolous fungi, Mycologia 89, 199-219.
9. Leonowicz, A., Matuszewska, A., Luterek, J., Ziegenhagen, D., Wojtas-Wasilewska, M., Cho, N. S., Hofrichter, M., and Rogalski, J. (1999) Biodegradation of lignin by White Rot Fungi, Fungal Genet Biol 27, 175-185.
10. Buswell, J. A., and Odier, E. (1987) LIGNIN BIODEGRADATION, Crc Critical Reviews in Biotechnology 6, 1-60.
11. Gold, M. H., Wariishi, H., and Valli, K. (1989) EXTRACELLULAR PEROXIDASES INVOLVED IN LIGNIN DEGRADATION BY THE WHITE ROT BASIDIOMYCETE PHANEROCHAETE-CHRYSOSPORIUM, ACS Symp Ser 389, 127-140.
12. Kirk, T. K., and Farrell, R. L. (1987) ENZYMATIC COMBUSTION—THE MICROBIAL-DEGRADATION OF LIGNIN, Annu Rev Microbiol 41, 465-505.
13. Gold, M. H., and Alic, M. (1993) Molecular-biology of the lignin-degrading basidiomycete Phanerochaete-chrysosporium, Microbiol Rev 57, 605-622.
14. Bumpus, J. A., and Aust, S. D. (1987) BIODEGRADATION OF ENVIRONMENTAL-POLLUTANTS BY THE WHITE ROT FUNGUS PHANEROCHAETE-CHRYSOSPORIUM—INVOLVEMENT OF THE LIGNIN DEGRADING SYSTEM, BioEssays 6, 166-170.
15. Hammel, K. E. (1989) ORGANOPOLLUTANT DEGRADATION BY LIGNINOLYTIC FUNGI, Enzyme Microb Technol 11, 776-777.
16. Joshi, D. K., and Gold, M. H. (1993) DEGRADATION OF 2,4,5-TRICHLOROPHENOL BY THE LIGNIN-DEGRADING BASIDIOMYCETE PHANEROCHAETE-CHRYSOSPORIUM, Appl Environ Microbiol 59, 1779-1785.
17. Kelley, R. L., Ramasamy, K., and Reddy, C. A. (1986) Characterization of Glucose-Oxidase Negative Mutants of a Lignin Degrading Basidiomycete Phanerochaete-Chrysosporium, Arch Microbiol 144, 254-257.
18. Ramasamy, K., Kelley, R. L., and Reddy, C. A. (1985) Lack of Lignin Degradation by Glucose Oxidase-Negative Mutants of Phanerochaete-Chrysosporium, Biochem Biophys Res Commun 131, 436-441.
19. Formey, L. J., Reddy, C. A., Tien, M., and Aust, S. D. (1982) The Involvement of Hydroxyl Radical Derived from Hydrogen-Peroxide in Lignin Degradation by the White Rot Fungus Phanerochaete-Chrysosporium, J Biol Chem 257, 1455-1462.
20. Kelley, R. L., and Reddy, C. A. (1986) Identification of Glucose-Oxidase Activity as the Primary Source of Hydrogen-Peroxide Production in Ligninolytic Cultures of Phanerochaete-Chrysosporium, Arch Microbiol 144, 248-253.
21. Beach, R. L., Burton, W. V., Hendricks, W. J., and Festoff, B. W. (1982) Extracellular matrix synthesis by skeletal muscle in culture. Proteins and effect of enzyme degradation, J Biol Chem 257, 11437-11442.
22. Witt, S., Singh, M., and Kalisz, H. M. (1998) Structural and kinetic properties of nonglycosylated recombinant Penicillium amagasakiense glucose oxidase expressed in Escherichia coli, Appl Environ Microbiol 64, 1405-1411.
23. Das, A., and Kundu, P. N. (1987) Microbial-Production of Gluconic Acid, J Sci Ind Res 46, 307-311.
24. Sawyer, D. T. (1964) Metal-Gluconate Complexes, Chemical Reviews 64, 633-&.
25. Chun, U. H., and Rogers, P. L. (1988) The Simultaneous Production of Sorbitol from Fructose and Gluconic Acid from Glucose Using an Oxidoreductase of Zymomonas-Mobilis, Appl Microbiol Biotechnol 29, 19-24.
26. Cazetta, M. L., Celligoi, M., Buzato, J. B., and Scamino, I. S. (2007) Fermentation of molasses by Zymomonas mobilis: Effects of temperature and sugar concentration on ethanol production, Bioresour Technol 98, 2824-2828.
27. N. Bardiya, P. K. T. Shiu, Cyclosporin A-resistance based gene placement system for Neurospora crassa. Fungal Genet Biol 44 (2007) 307-314.
28. U. Baminger, S. S. Subramaniam, V. Renganathan, D. Haltrich, Purification and characterization of cellobiose dehydrogenase from the plant pathogen Sclerotium (Athelia) rolfsii. Appl Environ Microbiol 67 (2001) 1766-1774.
29. Z. Fan, G. B. Oguntimein, P. J. Reilly, Characterization of kinetics and thermostability of Acremonium strictum glucooligosaccharide oxidase. Biotechnol Bioeng 68 (2000) 231-237.
30. H. J. Vogel, A convenient growth medium for Neurospora. Microbiol Genet Bull 13 (1956) 42-46.
31. J. Sambrook, E. F. Fritsch, T. Maniatis, Molecular Cloning: A Laboratory Manual, Cold Spring Harbor Laboratory Press, New York, 1989.
32. M. M. Bradford, Rapid and Sensitive Method for Quantitation of Microgram Quantities of Protein Utilizing Principle of Protein-Dye Binding. Anal Biochem 72 (1976) 248-254.

All of the references discussed herein are hereby incorporated by reference, in their entireties.

1. A method for the production of sugar acids and sugar salts from cellulosic materials, the method comprising:
a) selecting one or more microorganisms that are capable of expressing and secreting enzymes, which hydrolyze cellulosic materials, wherein said one or more microorganisms are genetically modified microorganisms that exhibit a reduced level of β-glucosidase or β-xylosidase activity as compared to wild-type;
b) combining said one or more microorganisms in an aerobic solution with cellulosic materials to facilitate conversion of said cellulosic materials into one or more sugars acids;
c) precipitating said one or more sugar acids or sugar salts from solution.

2. The method of claim 1, wherein said one or more precipitated sugar acids or sugar acid salts are combined with β-glucosidase or β-xylosidase in an anaerobic solution to facilitate fermentation of said one or more sugars acids or sugar salts to form one or more chemicals.

3. The method of claim 1, wherein said cellulosic materials is selected from the group consisting of lignin, and/or, cellulose, and/or, hemicellulose.

4. The method of claim 1, wherein said one or more microorganisms is selected from the group consisting of bacteria, yeast or fungi.

5. The method of claim 1, wherein said one or more microorganisms is selected from the group consisting of White Rot Fungi, Brown Rot Fungi, Soft Rot Fungi, Trichoderma or Aspergillus.

6. The method of claim 1, wherein said one or more microorganisms are capable of secreting enzymes selecting from the group consisting of cellulases, xylanases, ligninases, oxidases, laccases, catalases, and dehydrogenases.

7. The method of claim 1, wherein said one or more sugars is a polymer comprised of 2-10 units.

8. The method of claim 1, wherein said one or more sugars is selected from the group consisting of glucose, xylose, galactose, arabinose, and mannose.

9. The method of claim 1, wherein said one or more sugar acids or sugar salts is saccharide aldonic acid (SAA) or a salt of saccharide aldonic acid.

10. The method of claim 1, wherein a revised form of lignin is produced.

11. The method of claim 1, wherein the pH of said aerobic solution is 2 to 12.

12. The method of claim 1, wherein the temperature of said aerobic solution is 20° C. to 80° C.

13-23. (canceled)

24. The method of claim 1, wherein said one or more microorganisms over-express cellobiose dehydrogenase (CDH) or xylobiose dehydrogenase (XDH), as compared to wild-type, to convert said one or more sugars into corresponding sugar acids or sugar salts.

25-36. (canceled)

37. The method of claim 1, wherein said aerobic solution includes exogenous cellobiose dehydrogenase (CDH) and/or xylobiose dehydrogenase (XDH).

38. The method of claim 1, blocking or lowering the expression level of one or more catabolite repression proteins.

39. A method for the production of sugar acids and sugar salts from cellulosic materials, the method comprising:
d) selecting one or more microorganisms that are capable of expressing and secreting enzymes which hydrolyze cellulosic materials;
e) combining said one or more microorganisms in an aerobic solution with cellulosic materials to facilitate hydrolysis of said cellulosic materials into one or more sugars acids;
f) inhibiting the expression of β-glucosidase or β-xylosidase in said one or more microorganisms by gene knockdown or chemical inhibition to reduce the utilization of said one or more sugars by said one or more microorganisms;
g) precipitating said one or more sugar acids or sugar salts from solution.

40-77. (canceled)

78. A method for the production of sugar acids and sugar salts from cellulosic materials, the method comprising:
a) selecting one or more microorganisms that is capable of expressing and secreting enzymes which hydrolyze cellulosic materials, wherein said one or more microorganisms are genetically modified microorganisms that over-express cellobiose dehydrogenase (CDH) and/or xylobiose dehydrogenase (XDH) as compared to wild-type;
b) combining said one or more microorganisms in an aerobic solution with cellulosic materials to facilitate hydrolysis of said cellulosic materials into one or more sugars;
c) precipitating said one or more sugar acids or sugar salts from solution.

79-102. (canceled)
